(19) United States
(12) Patent Application Publication
(10) Pub. No.: US 2010/0216727 A1
(43) Pub. Date: Aug. 26, 2010

(54) Carbohydrate-metallocene-antimalarial conjugates

(76) Inventors: Michael J. Adam, Surrey (CA); Chris Orvig, Vancouver (CA); Paloma Salas, Vancouver (CA); Christoph Herrmann, Haltern am See (DE)

Correspondence Address: HARNESS, DICKEY & PIERCE, P.L.C., P.O. BOX 8910, RESTON, VA, 20195, US

(21) Appl. No.: 12/656,946

(22) Filed: Feb. 22, 2010

Related U.S. Application Data
(60) Provisional application No. 61/202,340, filed on Feb. 20, 2009.
(60) Provisional application No. 61/304,598, filed on Feb. 15, 2010.

(51) Int. Cl. A61K 31/70; C07H 1/00; C08B 37/00; A61P 33/06

(52) U.S. Cl. 514/23; 536/1.11; 536/55

(57) ABSTRACT

An antimalarial conjugate according to a non-limiting embodiment of the present invention may include a metallocene, a carbohydrate, and an antimalarial agent. The metallocene may include two cyclopentadienyl rings bound to a central metal atom. The carbohydrate and the antimalarial agent may be appended to at least one of the cyclopentadienyl rings of the metallocene, wherein the antimalarial agent has therapeutic properties directed to treating and/or preventing malaria. The metallocene may be ferrocene, the carbohydrate may be glucose, and the antimalarial agent may be chloroquine.

### CROSS-REFERENCE TO RELATED APPLICATION

This application claims priority under 35 U.S.C. §119(e) to U.S. Provisional Application No. 61/202,340, filed Feb. 20, 2009, and U.S. Provisional Application No. 61/304,598, filed Feb. 15, 2010, the entire contents of each of which are incorporated herein by reference.

### BACKGROUND

1. Field

Example embodiments of the present invention relate to antimalarial compounds.

2. Description of Related Art

Malaria is a widespread parasitic disease that affects a relatively large population in tropical and subtropical regions. At least four species of the human infecting parasite have been identified, with Plasmodium falciparum being the most lethal. It is estimated that 2 billion people have been exposed to the parasite. About 300-500 million cases of malaria are reported each year, with 1 to 2.7 million people dying from the infection annually. Furthermore, global warming may facilitate the spread of malaria into more temperate regions where the disease has not previously been a health concern. Although a variety of drugs are available to treat malaria, resistance has developed with regard to several of the antimalarial drugs, such as chloroquine. As a result, such drug resistance has diminished the ability to effectively combat malaria.

### SUMMARY

An antimalarial conjugate according to a non-limiting embodiment of the present invention may include a metallocene including two cyclopentadienyl rings bound to a central metal atom; a carbohydrate appended to the metallocene; and an antimalarial agent appended to the metallocene, wherein the antimalarial agent has therapeutic properties directed to treating and/or preventing malaria.

An antimalarial conjugate according to another non-limiting embodiment of the present invention may include a metallocene including two cyclopentadienyl rings bound to a central metal atom; and a plurality of antimalarial agents appended to the metallocene, wherein the antimalarial agents have therapeutic properties directed to treating and/or preventing malaria.

### BRIEF DESCRIPTION OF THE DRAWINGS

FIGS. 1A-1B illustrate a metallocene and a ferrocene, respectively, for an antimalarial conjugate according to a non-limiting embodiment of the present invention.

FIG. 2 illustrates derivatives of glucose for an antimalarial conjugate according to a non-limiting embodiment of the present invention.

FIG. 3 illustrates linkers for an antimalarial conjugate according to a non-limiting embodiment of the present invention.

FIGS. 4A-4B illustrate 1,2-homoannular arrangements and 1,1′-heteroannular arrangements, respectively, for an antimalarial conjugate according to a non-limiting embodiment of the present invention.

FIG. 5 illustrates antimalarial conjugates wherein the ferrocene is in a terminal position with respect to the chloroquine according to a non-limiting embodiment of the present invention.

FIGS. 6A-6C illustrate antimalarial conjugates wherein the ferrocene is in an internal position with respect to the chloroquine according to a non-limiting embodiment of the present invention.

FIG. 7 illustrates a synthesis outline for carbohydrate-metallocene-antimalarial conjugates according to a non-limiting embodiment of the present invention.

FIG. 8 illustrates another synthesis outline for carbohydrate-metallocene-antimalarial conjugates according to a non-limiting embodiment of the present invention.

FIGS. 9A-9C illustrate a method of synthesizing a 1,2-substituted conjugate according to a non-limiting embodiment of the present invention.

FIG. 10 illustrates the preparation of a starting material in connection with the synthesis of a 1,2-substituted conjugate according to a non-limiting embodiment of the present invention.

FIG. 11 illustrates functionalization with a carbohydrate and deprotection in connection with the synthesis of a 1,2-substituted conjugate according to a non-limiting embodiment of the present invention.

FIG. 12 illustrates reductive amination in connection with the synthesis of a 1,2-substituted conjugate according to a non-limiting embodiment of the present invention.

FIG. 13 illustrates the formation of a linker in connection with the synthesis of a 1,2-substituted conjugate according to a non-limiting embodiment of the present invention.

FIGS. 14A-14C illustrate a method of synthesizing a 1,1′-substituted conjugate according to a non-limiting embodiment of the present invention.

FIG. 15 illustrates the preparation of a starting material in connection with the synthesis of a 1,1′-substituted conjugate according to a non-limiting embodiment of the present invention.

FIG. 16 illustrates antimalarial conjugates including a metallocene and a plurality of antimalarial agents according to a non-limiting embodiment of the present invention.

FIG. 17 illustrates a method of synthesizing 1,1′-substituted conjugates including a plurality of antimalarial agents according to a non-limiting embodiment of the present invention.

FIG. 18 illustrates another method of synthesizing a 1,1′-substituted conjugate including a plurality of antimalarial agents according to a non-limiting embodiment of the present invention.

FIG. 19 illustrates another method of synthesizing a 1,1′-substituted conjugate including a plurality of antimalarial agents according to a non-limiting embodiment of the present invention.

### DETAILED DESCRIPTION

It will be understood that when an element or structure is referred to as being “connected,” “coupled,” “bonded,” or “appended” to another element or structure, it may be directly connected, coupled, bonded, or appended to the other element or structure or intervening elements or structures may be present. In contrast, when an element or structure is referred to as being “directly connected,” “directly coupled,” “directly bonded,” or “directly appended” to another element or structure, there are no intervening elements or layers present. Like numbers refer to like elements throughout the specification. As used herein, the term “and/or” includes any and all combinations of one or more of the associated listed items.

It will be understood that, although the terms first, second, third, etc. may be used herein to describe various elements and/or structures, these elements and/or structures should not be limited by these terms. These terms are only used to distinguish one element or structure from another element or structure. Thus, a first element or structure discussed below could be termed a second element or structure without departing from the teachings of example embodiments.

Spatially relative terms, e.g., “beneath,” “below,” “lower,” “above,” “upper,” and the like, may be used herein for ease of description to describe one element or structure's relationship to another element or structure as illustrated in the figures. It will be understood that the spatially relative terms are intended to encompass different orientations in addition to the orientation(s) depicted in the figure(s). For example, if the structure in the figures is turned over, elements described as “below” or “beneath” other elements or features would then be oriented “above” the other elements or features. Thus, the term “below” may encompass both an orientation of above and below. The structure may be otherwise oriented (rotated 90 degrees or at other orientations) and the spatially relative descriptors used herein interpreted accordingly.

The terminology used herein is for the purpose of describing various embodiments only and is not intended to be limiting of example embodiments. As used herein, the singular forms “a,” “an,” and “the” are intended to include the plural forms as well, unless the context clearly indicates otherwise. It will be further understood that the terms, “comprises,” “comprising,” “includes,” and/or “including,” if used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art. It will be further understood that terms, including those defined in commonly used dictionaries, should be interpreted as having a meaning that is consistent with their meaning in the context of the relevant art and will not be interpreted in an idealized or overly formal sense unless expressly so defined herein.

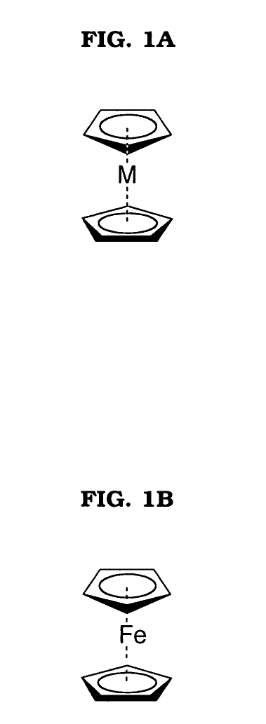

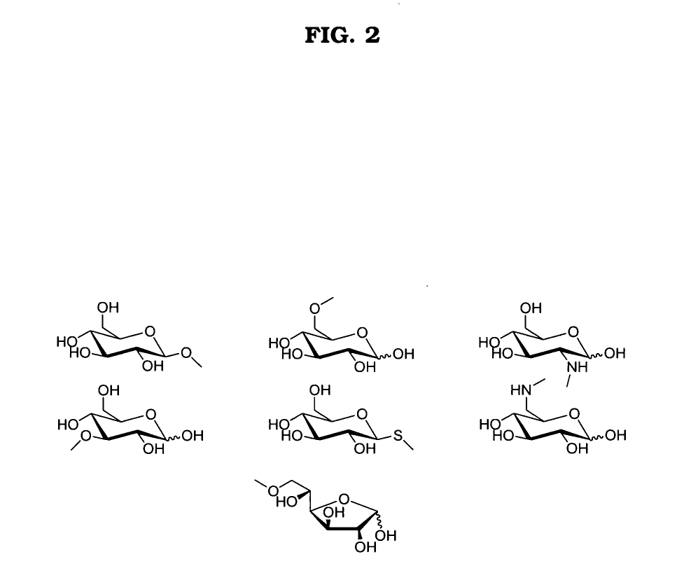

An antimalarial conjugate according to a non-limiting embodiment of the present invention may include a metallocene, a carbohydrate, and an antimalarial agent. Referring to FIG. 1A, the metallocene may include two cyclopentadienyl rings bound to a central metal atom M. The central metal atom may be an element selected from iron (Fe), ruthenium (Ru), and osmium (Os), although example embodiments are not limited thereto. For instance, the central metal atom may be one selected from Fe(II/III), Ga(III), Ru(II), Rh, Au(I), Os, Pd(II), and Pt(II)). In a non-limiting embodiment, the metallocene may be ferrocene, as illustrated in FIG. 1B.

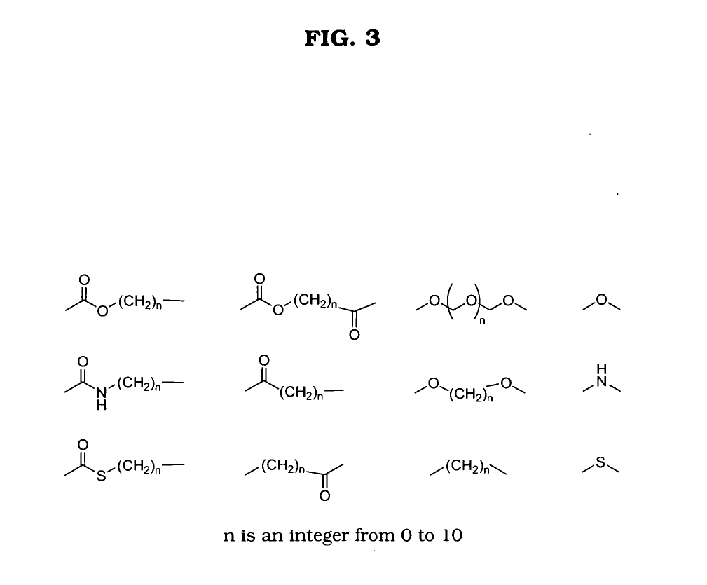

The carbohydrate may be appended to the metallocene. The carbohydrate may be a monosaccharide. The monosaccharide may be a hexose. In particular, the hexose may be an aldohexose (e.g., glucose, galactose). For example, when the carbohydrate is glucose, the glucose may be a derivative selected from the group shown in FIG. 2, although example embodiments are not limited thereto. Furthermore, a linker may be used to bind the metallocene to the carbohydrate. The linker may be selected from the group shown in FIG. 3, although example embodiments are not limited thereto.

The antimalarial agent may be appended to the metallocene. The antimalarial agent has therapeutic properties directed to treating and/or preventing malaria. The antimalarial agent may be a hemozoin inhibitor (e.g., chloroquine, mefloquine, quinine), an antifolate (e.g., sulfadoxine, pyrimethamine), and/or a sesquiterpene lactone (e.g., artemisinin).

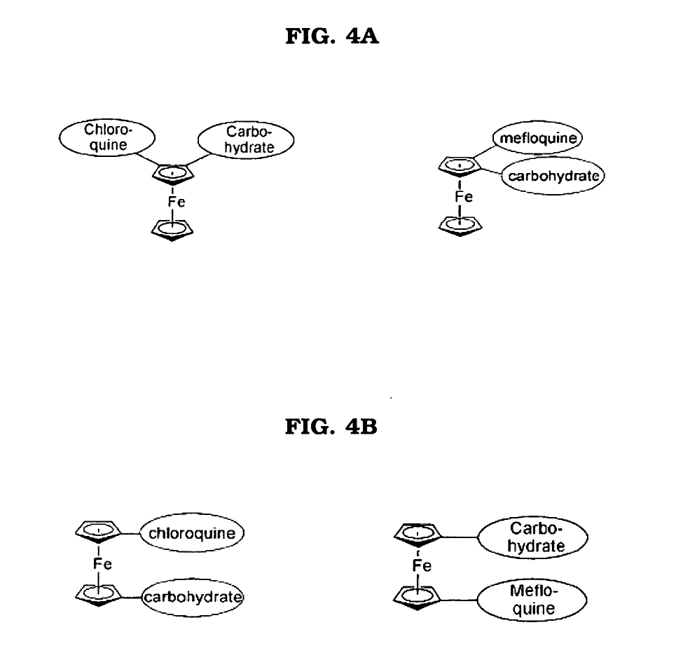

The carbohydrate and the antimalarial agent may be appended to the same cyclopentadienyl ring of the metallocene. For instance, the carbohydrate and the antimalarial agent may be appended to the cyclopentadienyl ring in a 1,2-homoannular fashion. Examples involving chloroquine-ferrocene-carbohydrate and mefloquine-ferrocene-carbohydrate conjugates are illustrated in FIG. 4A, although embodiments of the present invention are not limited thereto.

On the other hand, the carbohydrate and the antimalarial agent may be appended to different cyclopentadienyl rings of the metallocene. For instance, the carbohydrate and the antimalarial agent may be appended to the cyclopentadienyl rings in a 1,1′-heteroannular fashion. Examples involving chloroquine-ferrocene-carbohydrate and mefloquine-ferrocene-carbohydrate conjugates are illustrated in FIG. 4B, although embodiments of the present invention are not limited thereto.

Although not illustrated, it should be understood that other metallocenes and/or antimalarial agents may be used. Additionally, it should be understood that example embodiments also encompass conjugates involving other ring position combinations. It should also be understood that more than one carbohydrate and/or antimalarial agent may be appended to the upper and/or lower cyclopentadienyl rings of the metallocene. Where a plurality of carbohydrates and/or antimalarial agents are included in the conjugate, the carbohydrates and/or antimalarial agents may be identical compounds or different compounds.

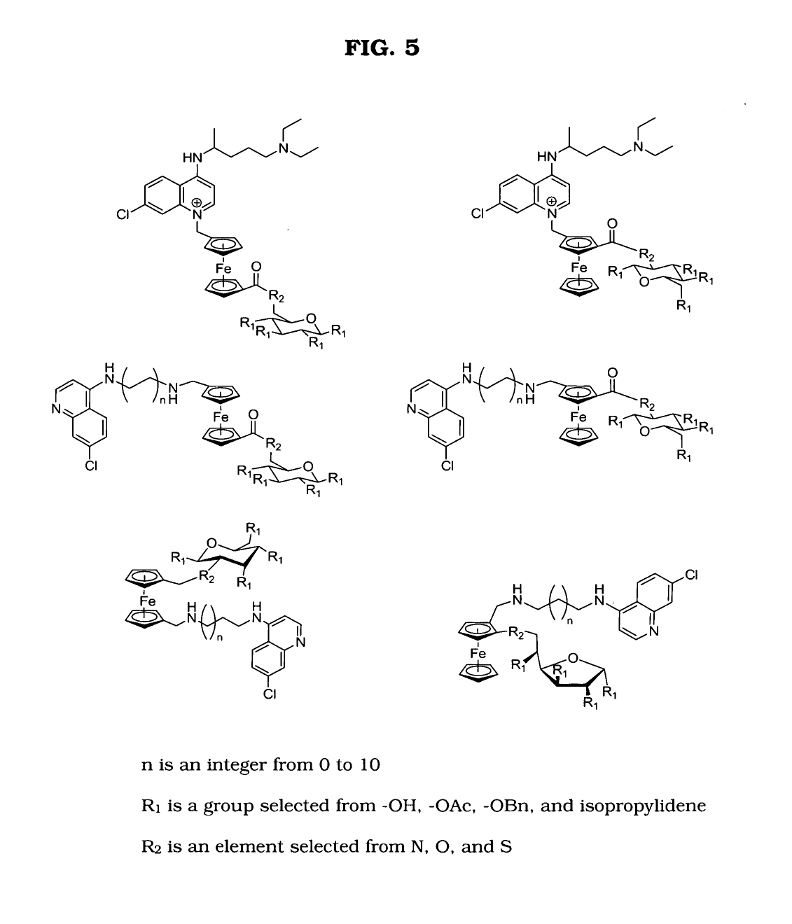

In a non-limiting embodiment, when the hemozoin inhibitor is chloroquine and the metallocene is ferrocene, the ferrocene may be in a terminal position with respect to the chloroquine. For instance, the antimalarial conjugate may be represented by one of the structures in FIG. 5, although example embodiments are not limited thereto.

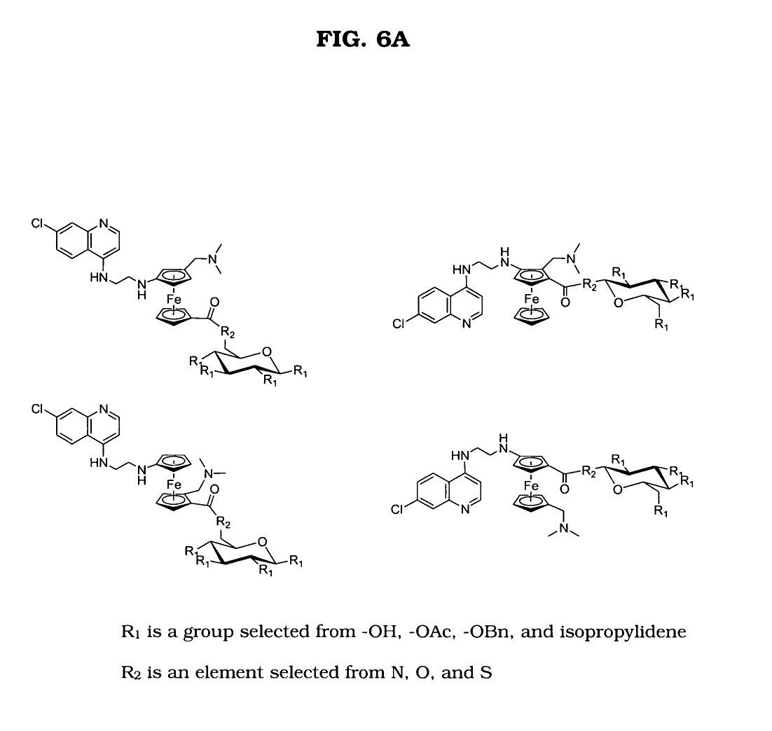

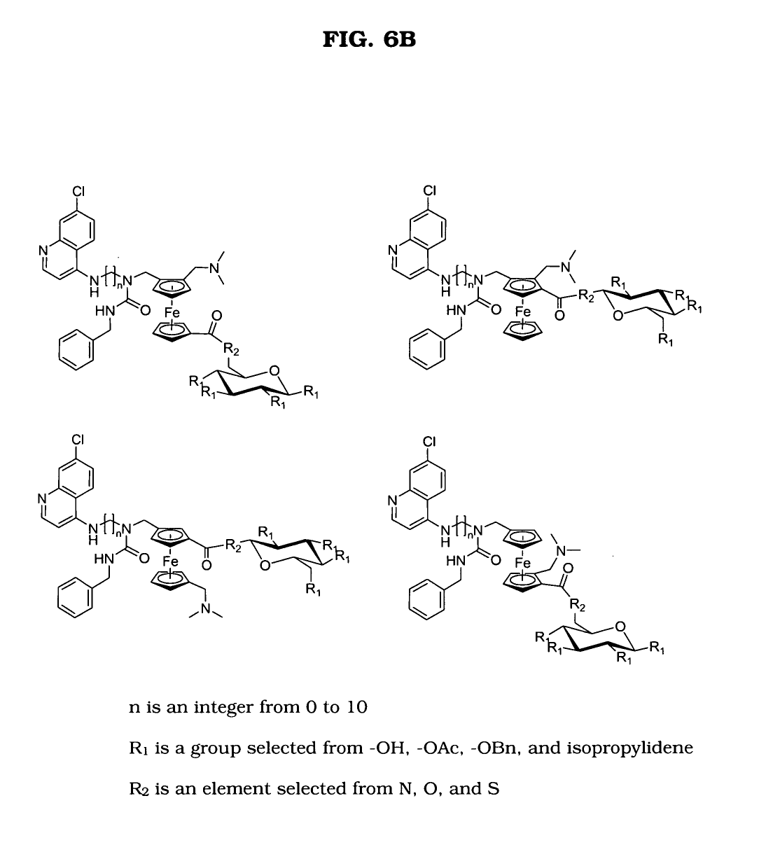

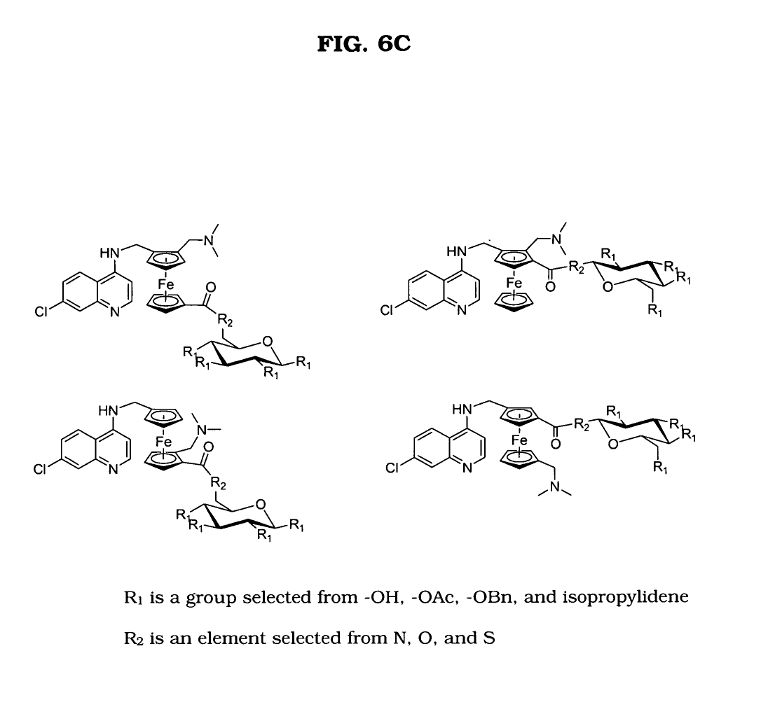

In another non-limiting embodiment, when the hemozoin inhibitor is chloroquine and the metallocene is ferrocene, the ferrocene may be in an internal position with respect to the chloroquine. For instance, the antimalarial conjugate may be represented by one of the structures in FIGS. 6A-6C, although example embodiments are not limited thereto.

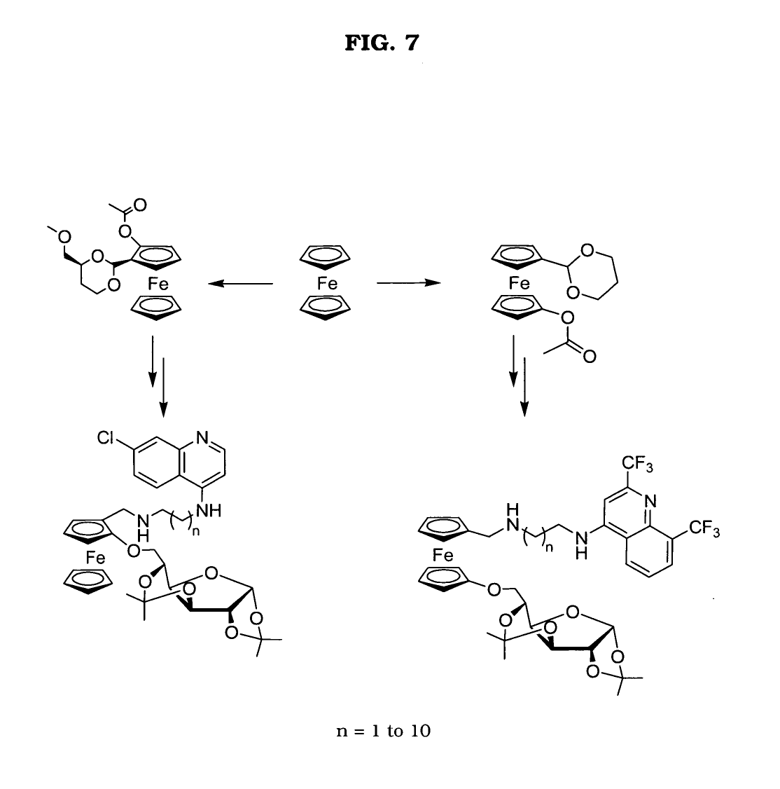

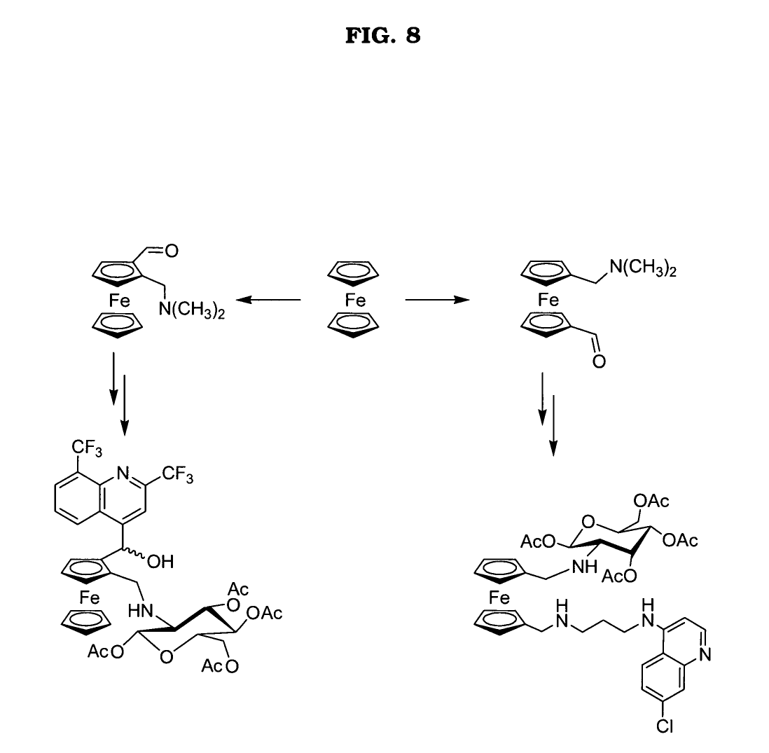

FIG. 7 illustrates a synthesis outline for carbohydrate-metallocene-antimalarial conjugates according to a non-limiting embodiment of the present invention. Referring to FIG. 7, the 1,2-substituted system includes a chloroquine derivative, while the 1,1′-substituted system includes a mefloquine derivative. FIG. 8 illustrates another synthesis outline for carbohydrate-metallocene-antimalarial conjugates according to a non-limiting embodiment of the present invention. Referring to FIG. 8, the 1,2-substituted system includes a mefloquine derivative, while the 1,1′-substituted system includes a chloroquine derivative. It should be understood that other carbohydrates, metallocenes, and/or antimalarial agents may be used in lieu of or included in addition to those shown in FIGS. 7-8.

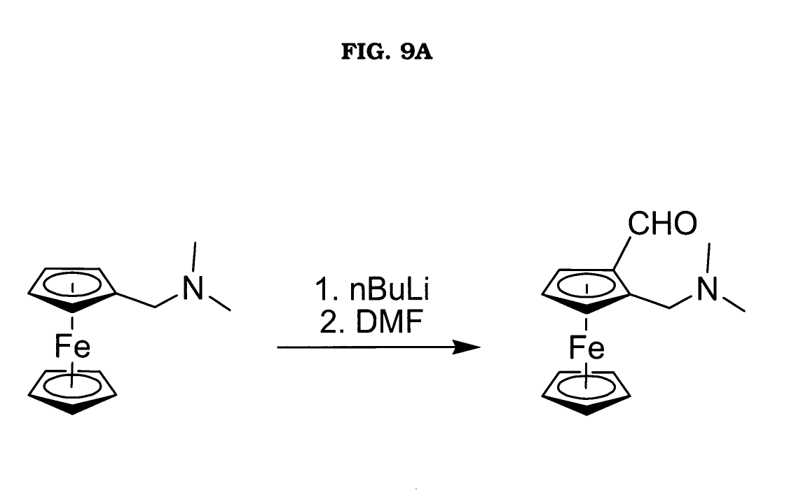

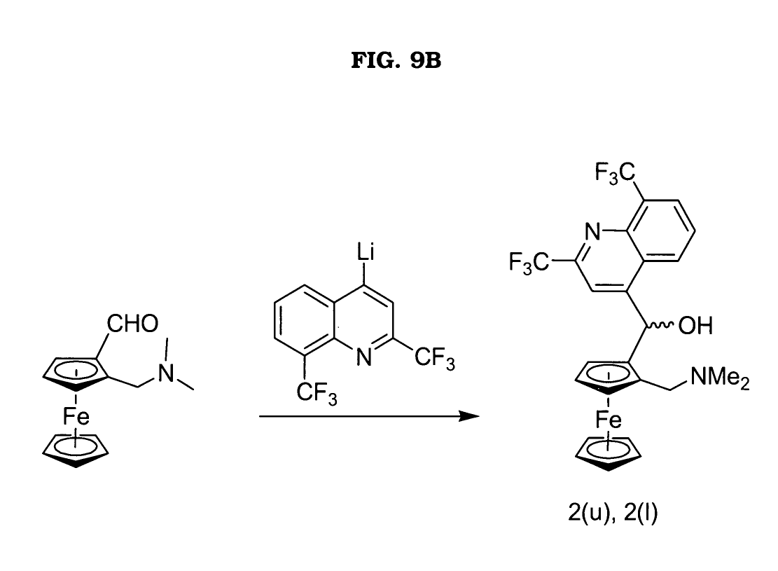

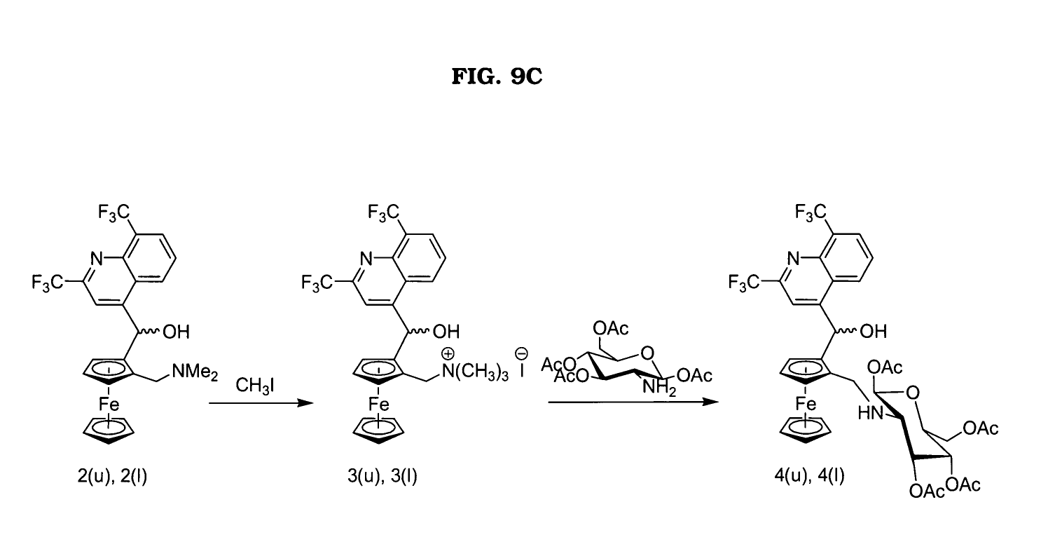

FIGS. 9A-9C illustrates a method of synthesizing a 1,2-substituted conjugate according to a non-limiting embodiment of the present invention. During synthesis, the functionalization of a cyclopentadienyl ring of the metallocene may be achieved as shown in FIG. 9A. Using the resulting structure from FIG. 9A, the coupling of an antimalarial agent may be achieved as shown in FIG. 9B. Using the resulting structure from FIG. 9B, the coupling of a carbohydrate may be achieved as shown in FIG. 9C. It should be understood that other carbohydrates, metallocenes, and/or antimalarial agents may be used in lieu of or included in addition to those shown in FIGS. 9A-9C.

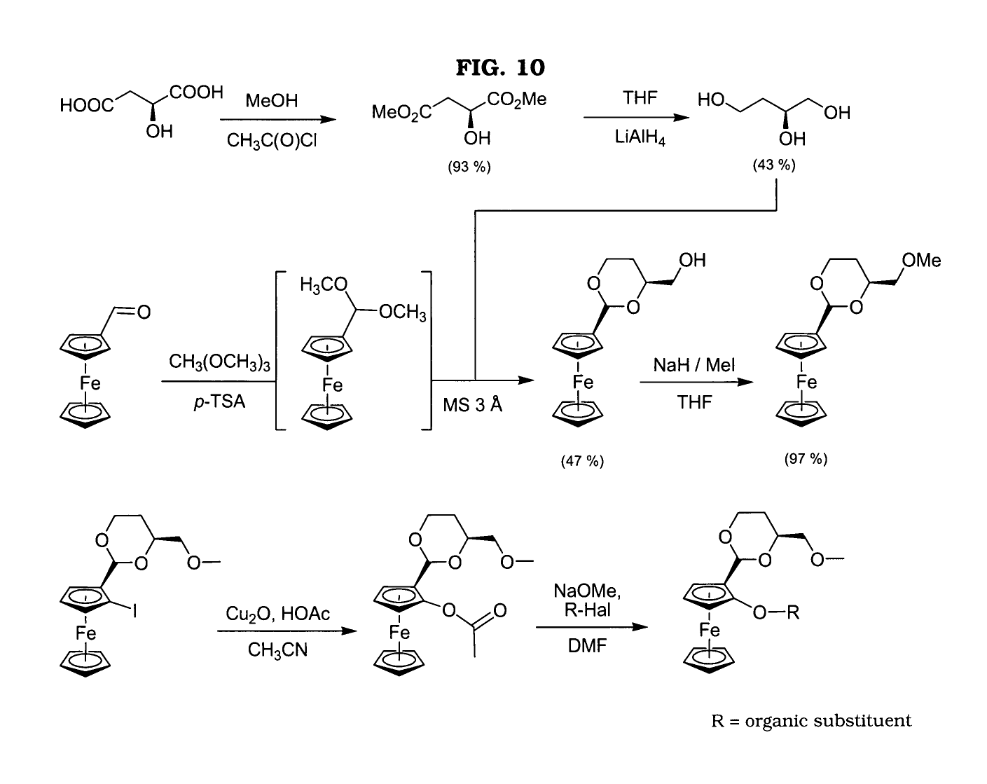

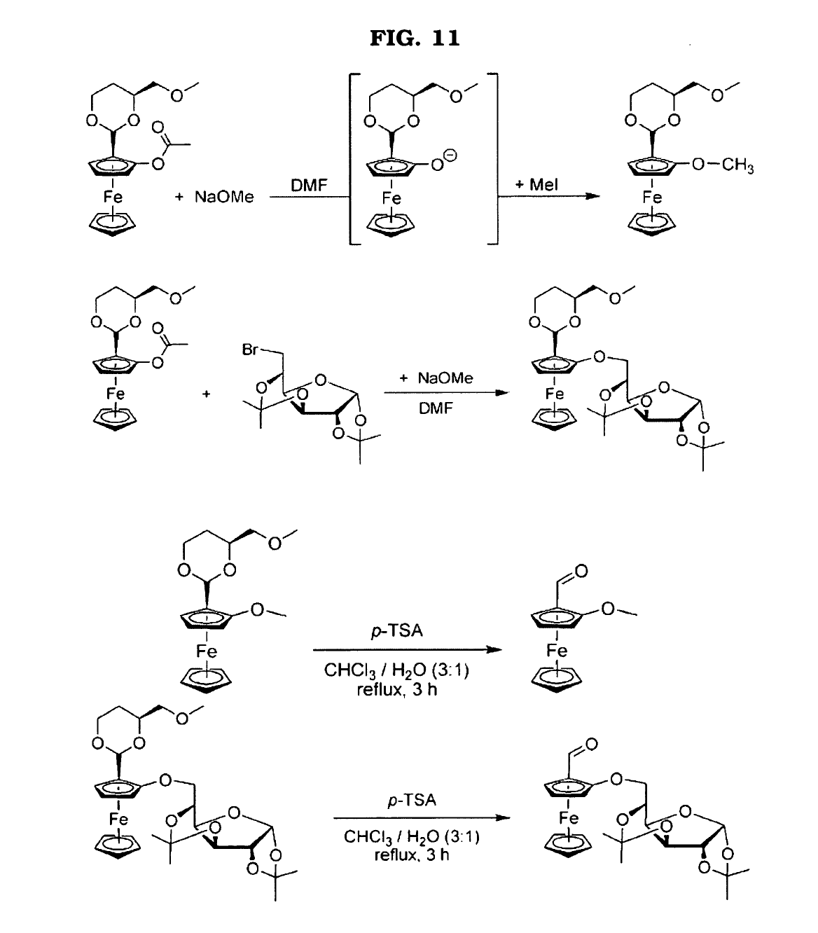

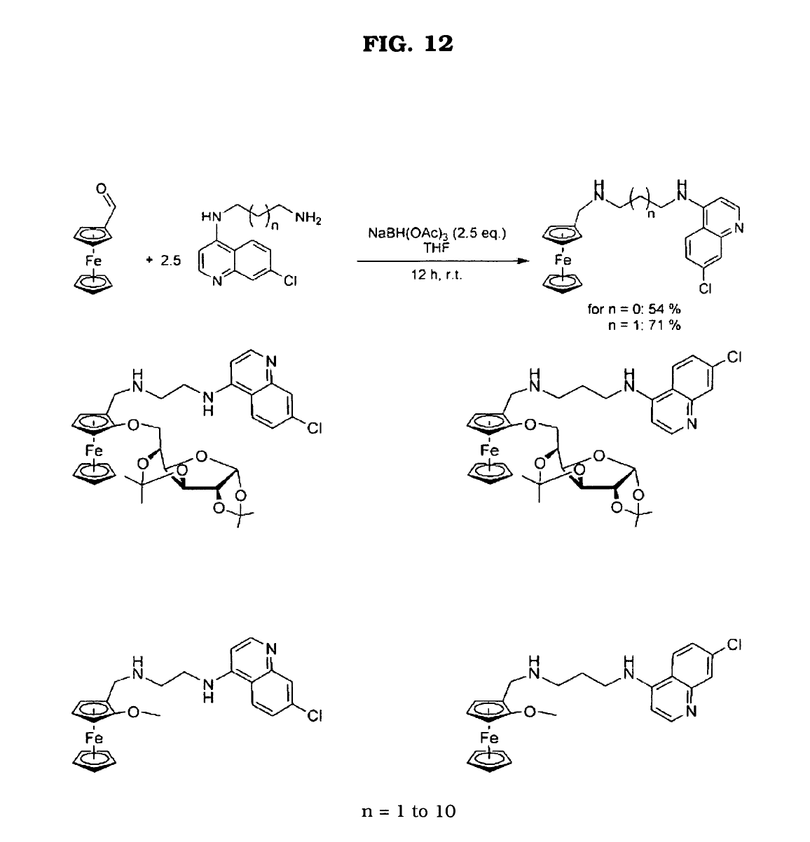

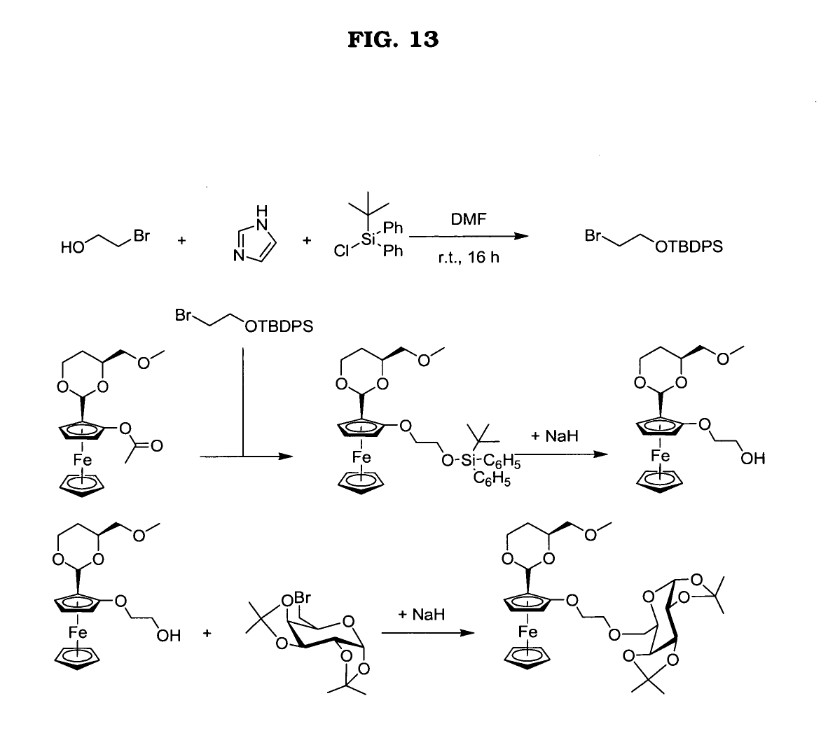

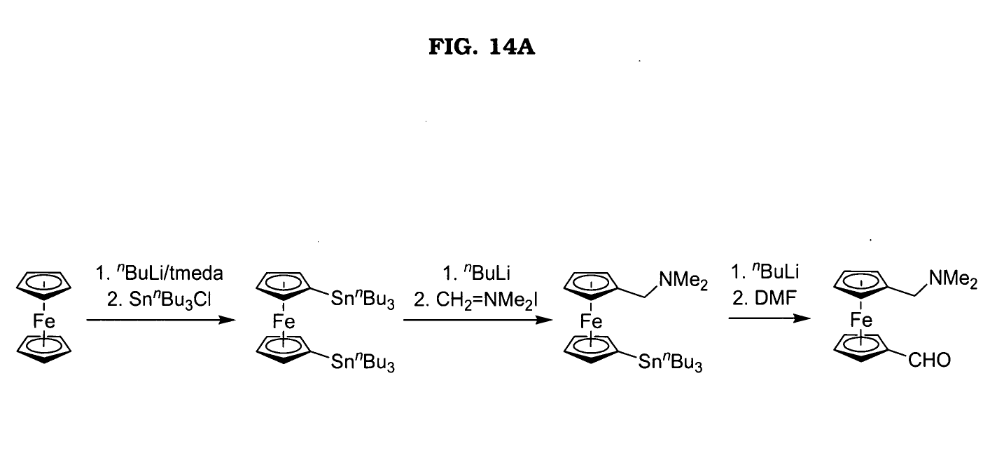

FIG. 10 illustrates the preparation of a starting material in connection with the synthesis of a 1,2-substituted conjugate according to a non-limiting embodiment of the present invention. Referring to FIG. 10, it should be understood that R may be any suitable organic substituent (e.g., alkyl, carbohydrate). FIG. 11 illustrates functionalization with a carbohydrate and deprotection in connection with the synthesis of a 1,2-substituted conjugate according to a non-limiting embodiment of the present invention. FIG. 12 illustrates reductive amination in connection with the synthesis of a 1,2-substituted conjugate according to a non-limiting embodiment of the present invention. FIG. 13 illustrates the formation of a linker in connection with the synthesis of a 1,2-substituted conjugate according to a non-limiting embodiment of the present invention.

In a non-limiting embodiment, 1,2-substituted antimalarial conjugates may be synthesized via a 6-step synthesis, beginning with ortho-substitution of N,N-dimethylamino ferrocene to afford the intermediate N,N′-dimethyl-2-ferrocenyl-carboxaldehyde which undergoes coupling reactions with 4-bromo-2,8-bis(trifluoromethyl)quinoline and 1,3,4,6-tetra-O-acetyl-2-amino-2-deoxy-β-D-glucose to yield the 4-[(2,8-bistrifluoromethyl)quinolyl]}methanol-1-[2-N-(1,3,4,6-tetra-O-acetyl-2-amino-2-deoxy-β-D-glucose)]-2-ferrocene conjugate. Alternatively, the 1,2-substituted antimalarial conjugates may be synthesized starting from (2S,4S,Sp)-(−)-1-acetoxy-2-(4-methoxymethyl-1,3-dioxan-2-yl)ferrocene, followed by coupling with, e.g., 6-bromo-deoxy-1,2;3,5-diisopropylidene glucofuranose to yield the carbohydrate-ferrocene intermediate structure. Reductive amination at the deprotected carboxaldehyde functionality, using chloroquine derivatives, yields the disubstituted conjugate.

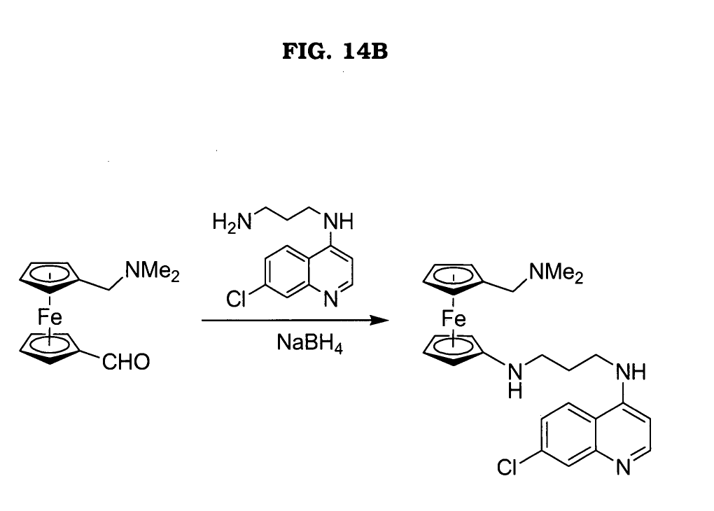

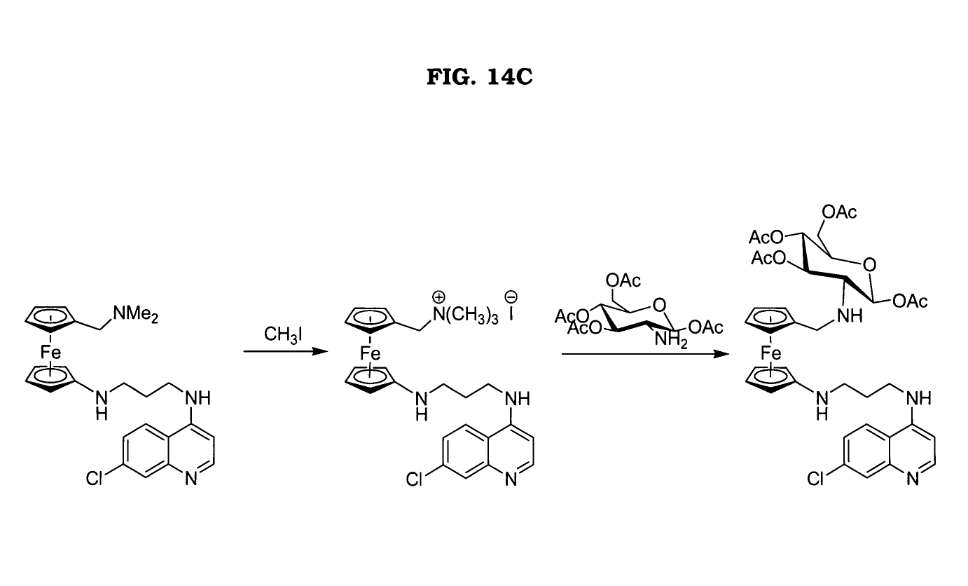

FIGS. 14A-14 illustrate a method of synthesizing a 1,1′-substituted conjugate according to a non-limiting embodiment of the present invention. During synthesis, the functionalization of the cyclopentadienyl rings of the metallocene may be achieved as shown in FIG. 14A. Using the resulting structure from FIG. 14A, the coupling of an antimalarial agent may be achieved as shown in FIG. 14B. Using the resulting structure from FIG. 14B, the coupling of a carbohydrate may be achieved as shown in FIG. 14C. It should be understood that other carbohydrates, metallocenes, and/or antimalarial agents may be used in lieu of or included in addition to those shown in FIGS. 14A-14C.

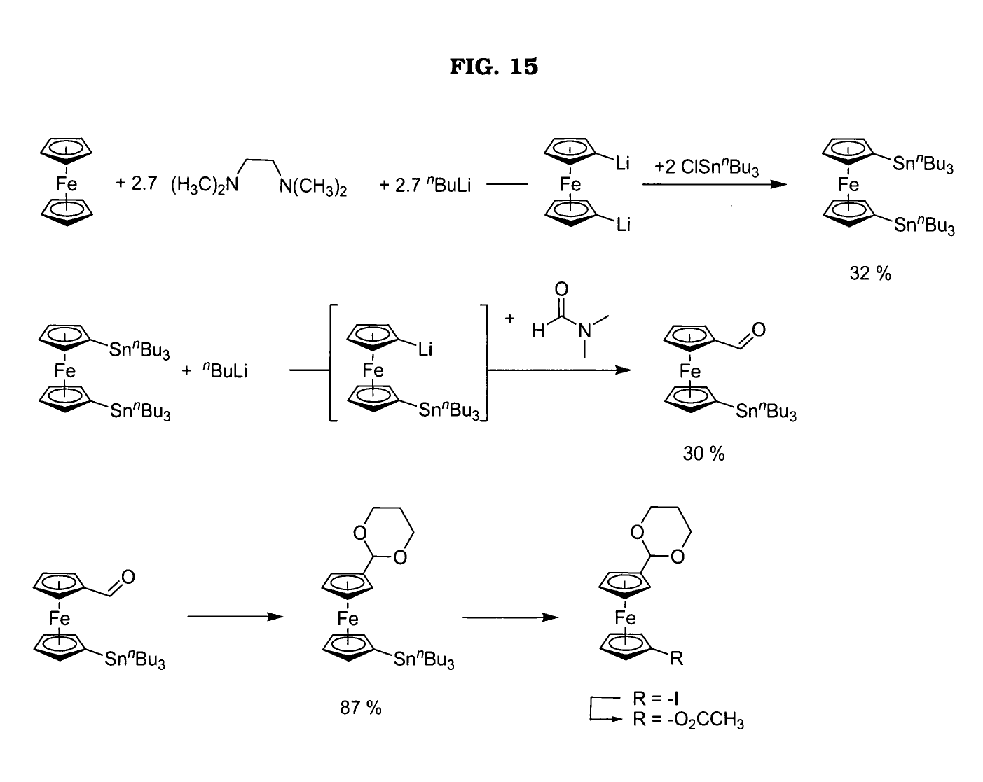

FIG. 15 illustrates the preparation of a starting material in connection with the synthesis of a 1,1′-substituted conjugate according to a non-limiting embodiment of the present invention. Functionalization with a carbohydrate and deprotection may be as disclosed in connection with FIG. 11. Additionally, reductive amination may be as disclosed in connection with FIG. 12. Furthermore, the formation of a linker may be as disclosed in connection with FIG. 13.

In a non-limiting embodiment, 1,1′-substituted conjugates may be synthesized starting via the selective functionalization of 1,1′-bis(tri-ⁿbutyl)tin ferrocene. Two different approaches may be employed. In one approach, 1-acetoxy-1′-(1,3-dioxan-2-yl) ferrocene is synthesized, which then can be functionalized in a similar manner as the 1,2-substituted derivatives, to yield the conjugate. In another approach, the heteroannular-substituted conjugate may be synthesized in a 7-step synthesis from the stannyl compound, via the intermediate 1′-(N,N-dimethylaminomethyl)-ferrocenyl-1-carboxaldehyde. Coupling reactions of the latter with the monosaccharide 1,3,4,6-tetra-O-acetyl-2-amino-2-deoxy-β-D-glucose and 7-Chloro-4-quinolinyl-(N′,N-dimethyl-1,4-pentanediamine) yield the conjugate 7-Chloro-4-[N-(4-N′,N′-diethylamino)-1-methylbutylamino]-1-[2-N-(1,3,4,6-tetra-O-acetyl-2-amino-2-deoxy-β-D-glucose)]-1′-amino ferrocene.

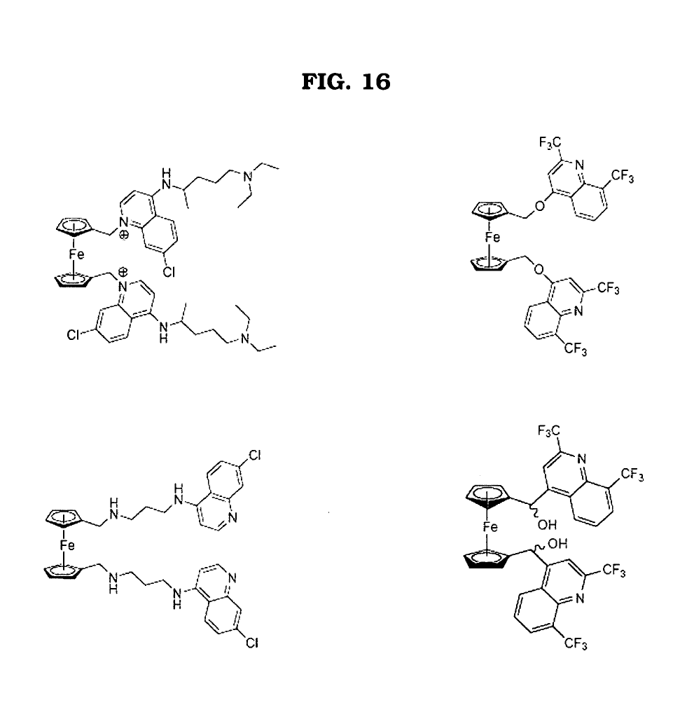

An antimalarial conjugate according to another non-limiting embodiment of the present invention may include a metallocene and a plurality of antimalarial agents but no carbohydrate. The metallocene may include two cyclopentadienyl rings bound to a central metal atom. The plurality of antimalarial agents may be appended to the metallocene, wherein the antimalarial agents have therapeutic properties directed to treating and/or preventing malaria. The antimalarial agents may be appended to the sa cyclopentadienyl ring of the metallocene. Alternatively, the antimalarial agents may be appended to different cyclopentadienyl rings of the metallocene. The antimalarial agents may be identical compounds or different compounds. Examples of such conjugates are shown in FIG. 16, although it should be understood that embodiments of the present invention are not limited thereto.

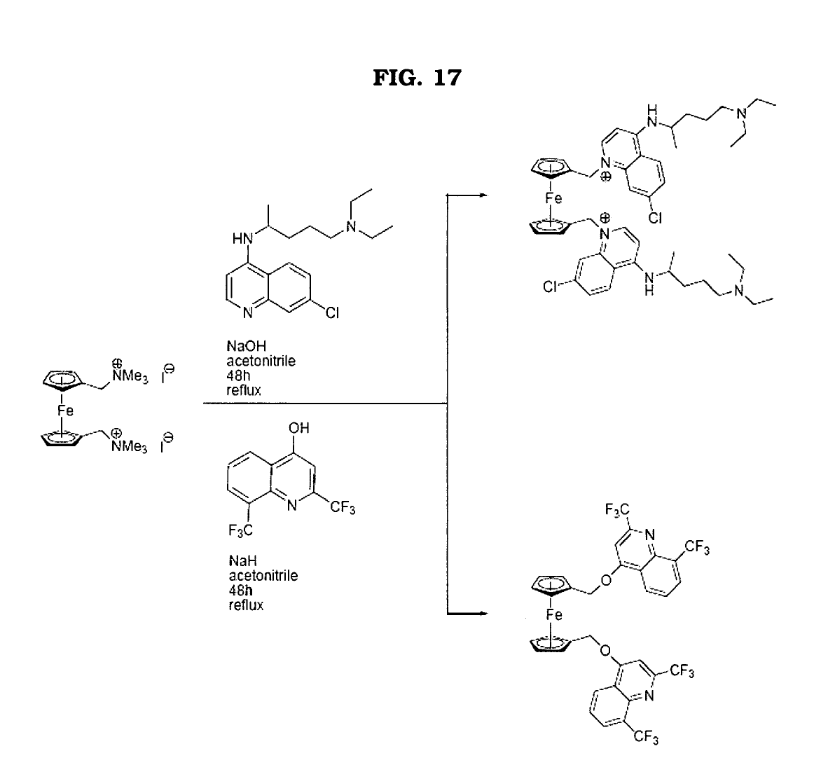

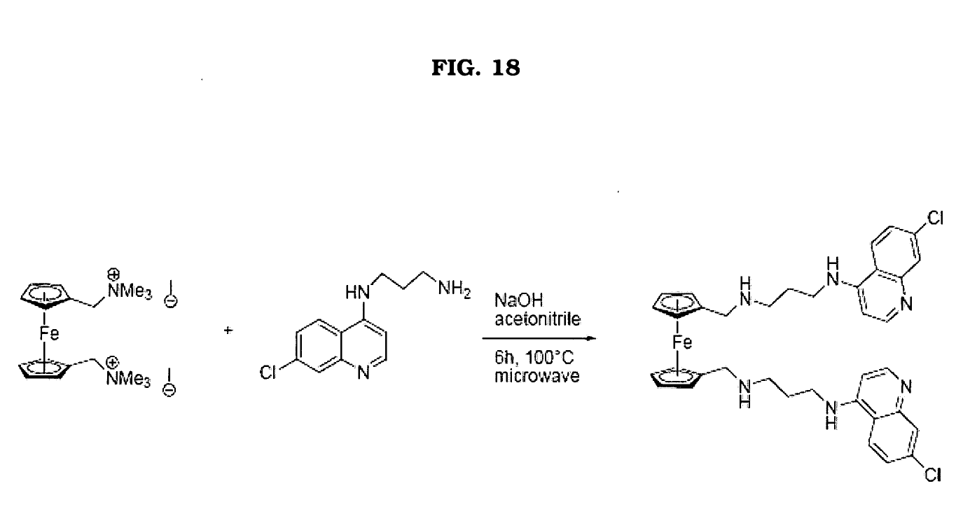

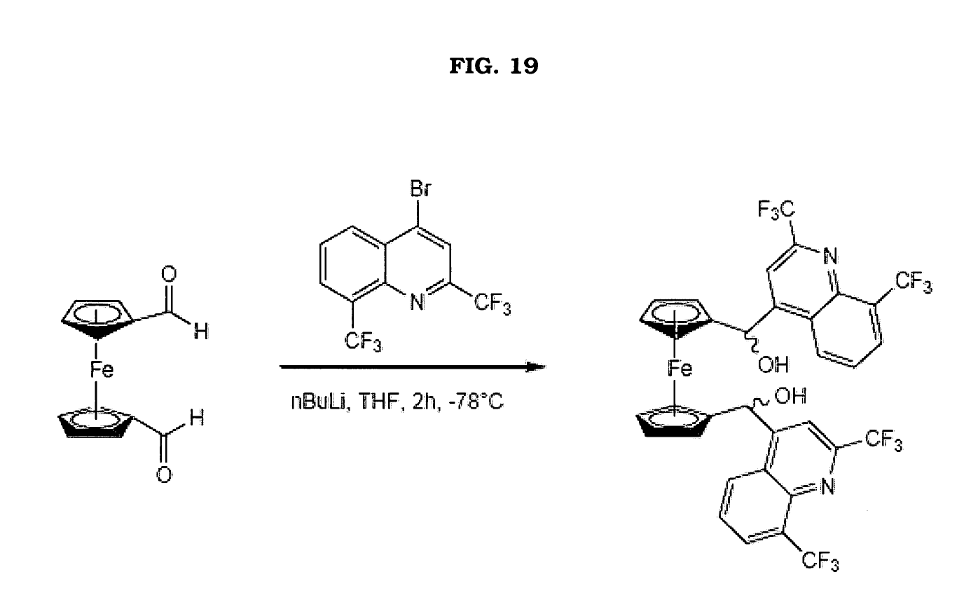

FIG. 17 illustrates a method of synthesizing 1,1′-substituted conjugates including a plurality of antimalarial agents according to a non-limiting embodiment of the present invention. FIG. 18 illustrates another method of synthesizing a 1,1′-substituted conjugate including a plurality of antimalarial agents according to a non-limiting embodiment of the present invention. FIG. 19 illustrates another method of synthesizing a 1,1′-substituted conjugate including a plurality of antimalarial agents according to a non-limiting embodiment of the present invention. Although not shown, it should be understood that a 1,2-substitution as well as other ring positions, metallocenes, and/or antimalarial agents may be employed.

Example embodiments of the present invention are directed to the treatment and/or prevention of malaria. A method of treating or preventing malaria may include administering an effective dosage of the antimalarial conjugate according to example embodiments to a patient in need thereof. The antimalarial conjugates according to example embodiments are relatively inexpensive to produce, a factor that will benefit developing countries. The conjugates also have activity in chloroquine-resistant parasite strains and have increased efficacy by targeting infected cells. Furthermore, because glucose uptake and metabolism in infected erythrocytes is elevated at all stages of the parasite's life cycle, conjugates including glucose may be particularly effective.

While example embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims.

1. An antimalarial conjugate, comprising:
a metallocene including two cyclopentadienyl rings bound to a central metal atom;
a carbohydrate appended to the metallocene; and
an antimalarial agent appended to the metallocene, the antimalarial agent having therapeutic properties directed to at least one of treating and preventing malaria.

2. The antimalarial conjugate of claim 1, wherein the central metal atom is an element selected from Fe, Ru, and Os.

3. The antimalarial conjugate of claim 1, wherein the carbohydrate and the antimalarial agent are appended to the same cyclopentadienyl ring of the metallocene.

4. The antimalarial conjugate of claim 3, wherein the carbohydrate and the antimalarial agent are appended to the cyclopentadienyl ring in a 1,2-homoannular fashion.

5. The antimalarial conjugate of claim 1, wherein the carbohydrate and the antimalarial agent are appended to different cyclopentadienyl rings of the metallocene.

6. The antimalarial conjugate of claim 5, wherein the carbohydrate and the antimalarial agent are appended to the cyclopentadienyl rings in a 1,1′-heteroannular fashion.

7. The antimalarial conjugate of claim 1, further comprising:
a linker selected from the following group: 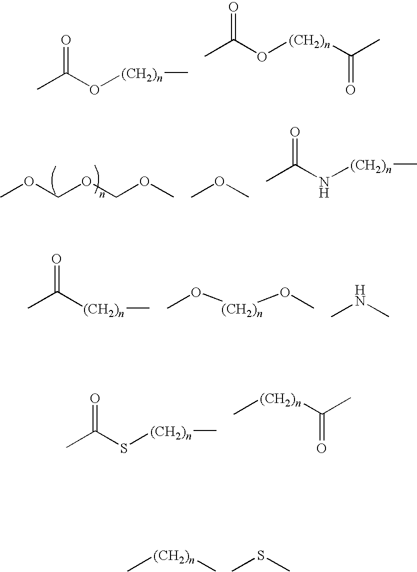
wherein the linker binds the metallocene to the carbohydrate, and n is an integer from 0 to 10.

8. The antimalarial conjugate of claim 1, wherein the carbohydrate is glucose or galactose.

9. The antimalarial conjugate of claim 8, wherein the glucose is a derivative selected from the following group:

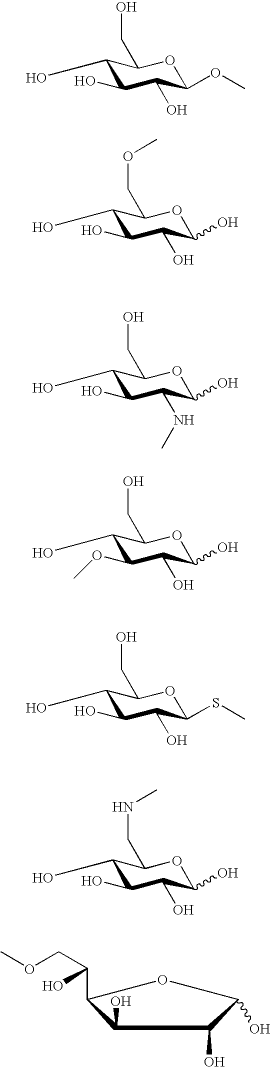

10. The antimalarial conjugate of claim 1, wherein the antimalarial agent is a hemozoin inhibitor.

11. The antimalarial conjugate of claim 10, wherein the hemozoin inhibitor is at least one of chloroquine, mefloquine, and quinine.

12. The antimalarial conjugate of claim 11, wherein the hemozoin inhibitor is chloroquine, the metallocene is ferrocene, and the ferrocene is in a terminal position with respect to the chloroquine.

13. The antimalarial conjugate of claim 12, wherein the antimalarial conjugate is represented by one of the following structures: 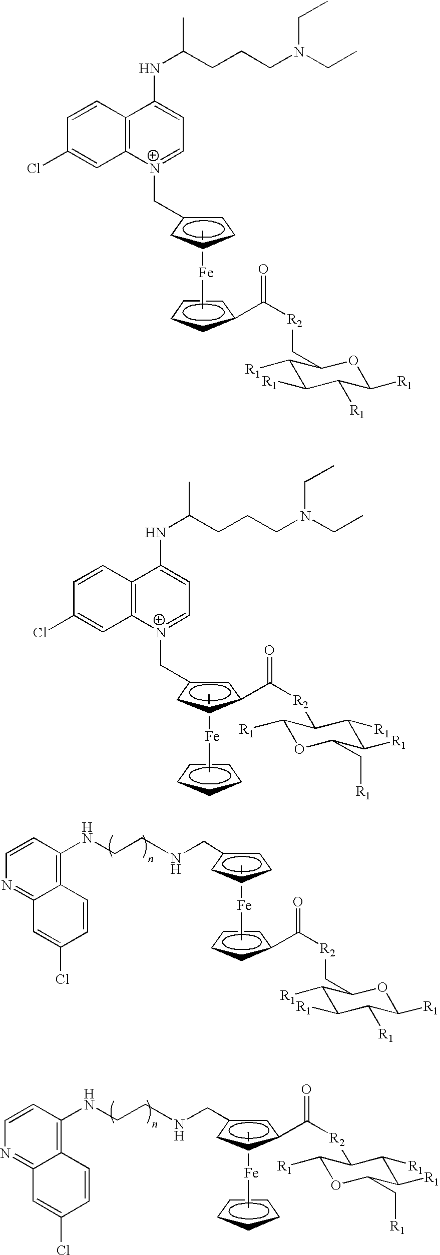 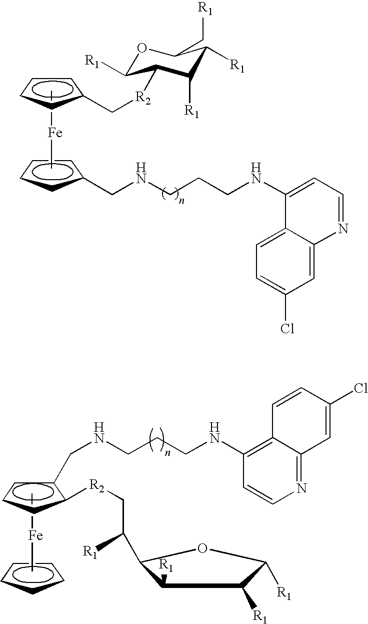
wherein
n is an integer from 0 to 10,
R₁ is a group selected from —OH, —OAc, -OBn, and isopropylidene,
R₂ is an element selected from N, O, and S.

14. The antimalarial conjugate of claim 11, wherein the hemozoin inhibitor is chloroquine, the metallocene is ferrocene, and the ferrocene is in an internal position with respect to the chloroquine.

15. The antimalarial conjugate of claim 14, wherein the antimalarial conjugate is represented by one of the following structures: 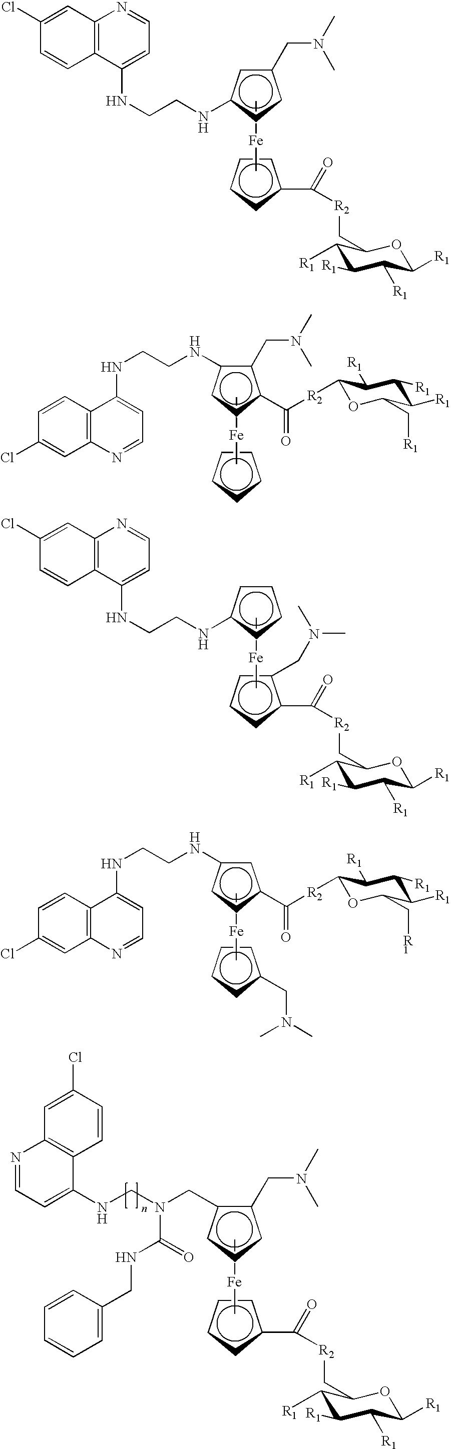 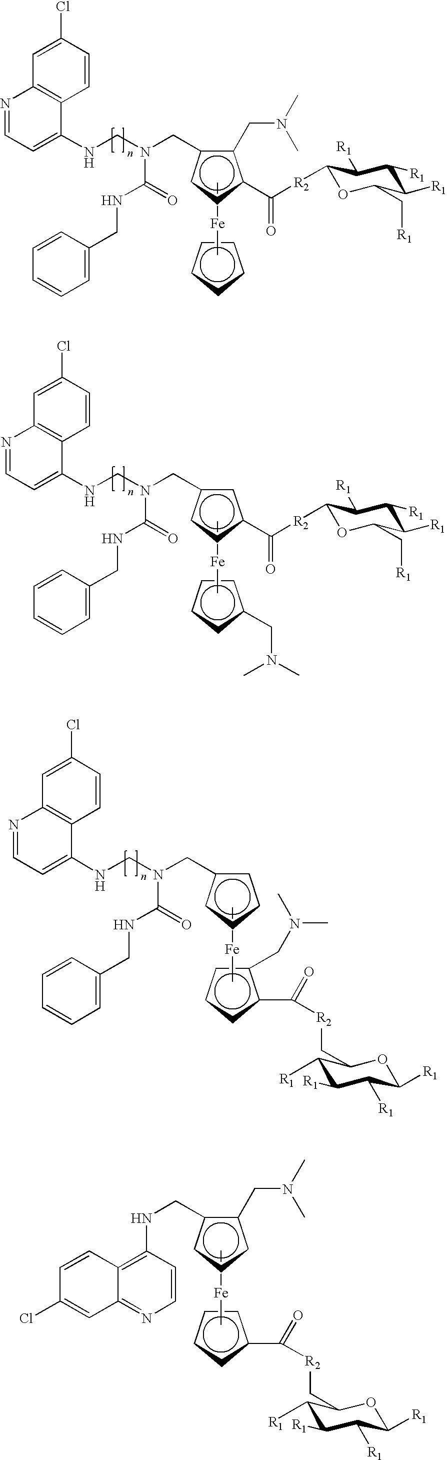 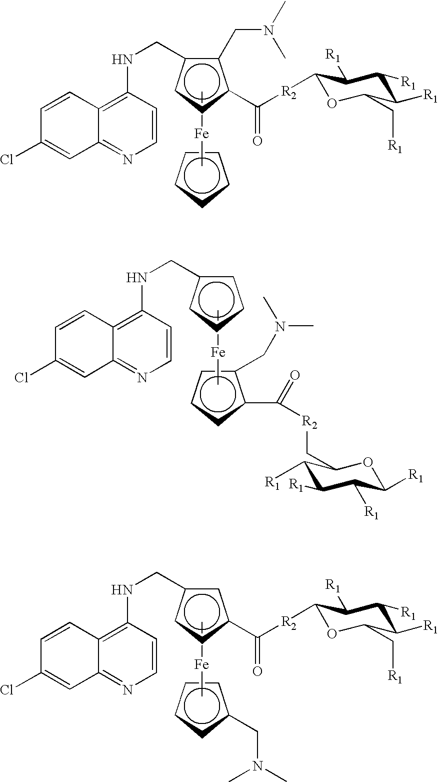
wherein
n is an integer from 0 to 10,
R₁ is a group selected from —OH, —OAc, -OBn, and isopropylidene,
R₂ is an element selected from N, O, and S.

16. The antimalarial conjugate of claim 1, wherein the antimalarial agent is an antifolate.

17. The antimalarial conjugate of claim 16, wherein the antifolate is at least one of sulfadoxine and pyrimethamine.

18. The antimalarial conjugate of claim 1, wherein the antimalarial agent is a sesquiterpene lactone.

19. The antimalarial conjugate of claim 18, wherein the sesquiterpene lactone is artemisinin.

20. A method of treating or preventing malaria, comprising:
administering an effective dosage of the antimalarial conjugate of claim 1 to a patient in need thereof.

21. An antimalarial conjugate, comprising:
a metallocene including two cyclopentadienyl rings bound to a central metal atom; and
a plurality of antimalarial agents appended to the metallocene, the antimalarial agents having therapeutic properties directed to at least one of treating and preventing malaria.

22. The antimalarial conjugate of claim 21, wherein the plurality of antimalarial agents includes two antimalarial agents appended to the same cyclopentadienyl ring of the metallocene.

23. The antimalarial conjugate of claim 21, wherein the plurality of antimalarial agents includes two antimalarial agents appended to different cyclopentadienyl rings of the metallocene.

24. The antimalarial conjugate of claim 21, wherein the plurality of antimalarial agents are identical compounds.

25. The antimalarial conjugate of claim 21, wherein the plurality of antimalarial agents are different compounds.

26. The antimalarial conjugate of claim 21, wherein the antimalarial conjugate is represented by one of the following structures: [chemical structure]
